(19) United States
(12) Patent Application Publication
(10) Pub. No.: US 2005/0165371 A1
(43) Pub. Date: Jul. 28, 2005

(54) Composite absorbent structure for the production of diapers, sanitary napkins and associated production method

(76) Inventor: Claudio Giacometti, Pistona (IT)

Correspondence Address: BREINER & BREINER, 115 NORTH HENRY STREET, P. O. BOX 19290, ALEXANDRIA, VA, 22314, US

(21) Appl. No.: 10/503,543

(22) PCT Filed: Jan. 30, 2003
(86) PCT No.: PCT/IT03/00038

(30) Foreign Application Priority Data
Feb. 5, 2002 (EP) 02425055.7

(51) Int. Cl. A61F 13/15; A61F 13/20

(52) U.S. Cl. 604/367

(57) ABSTRACT

An absorbent structure is described, said structure comprising: a first acquisition and distribution layer (S1) formed by a nonwoven fabric consisting of non-absorbent fibers; a second absorbent layer (s2) formed by a nonwoven fabric consisting of absorbent fibers and comprising particles (P) of at least one superabsorbent polymer distributed in the nonwoven fabric, in which at least said second layer is formed by staple fibers.

### TECHNICAL FIELD

The present invention relates to a composite absorbent structure for the production of hygiene articles such as female sanitary napkins, diapers for babies, diapers for incontinent persons and similar articles.

The invention also relates to a method for the production of a structure of the abovementioned type.

### BACKGROUND ART

In the production of absorbent hygiene articles it is known to provide complex structures comprising at least two outer layers, forming two containing shells, between which further layers with various functions, including usually a layer for acquisition and distribution of the body fluids and an absorbent layer, are arranged. Typically, a sanitary napkin or a diaper comprises a top sheet which is perforated or in any case permeable to liquids and intended to come into contact with the body of the person wearing it. The body fluids (blood, urine) intended to be absorbed by the article pass through the top sheet and reach the inside of the article. The top sheet is typically formed by a perforated plastic film and/or by a layer of nonwoven fabric or also by multiple-layer structures composed, for example of one or more plastic films joined to one or more strips or webs of textile fibers. An acquisition and distribution layer, referred to in short as “ADL”, is arranged underneath this top sheet. The acquisition and distribution layer ensures that the body liquids pass rapidly inside the structure of the napkin and distributes them so that they are distributed uniformly throughout the thickness of the underlying storage layer or core, instead of being absorbed in a localized manner only in the zones located underneath the points where the liquid arrives, or mainly in these zones. Underneath the storage layer where the liquids are absorbed and stored, there is located a backsheet which is impermeable to liquids and intended to prevent the absorbed liquids from escaping.

The various component layers of this structure are welded together normally along the edges of the article.

In special cases, even more complex structures, with a greater number of layers, are used.

In the production of this type of article the problem arises of having to assemble different starting materials provided in the form of continuous strips, using extremely costly machinery. It would therefore be convenient to have semifinished products consisting of strip-like materials which perform more than one of the functions listed above (i.e. top sheet, distribution, absorption, rear closure of the article), in order to reduce the complexity of the production machinery.

A further problem which arises during the production of absorbent articles of the type mentioned above consists in the volume of the articles themselves. In particular in the production of absorbent articles for female use it would be convenient to have materials of limited thickness, in order to obtain thin layers and hence reduce the discomfort when wearing them.

In order to solve at least partially the abovementioned problems, composite materials, consisting of two or more assembled layers which constitute a structure forming a semifinished starting product used to produce the finished article, have been studied.

U.S. Pat. No. 4,287,251 describes a disposable absorbent structure made from a plurality of layers of nonwoven fabric, which are for example entangled. These layers are joined together by means of a binding agent, in particular applied along the edges, or by means of spot welding using thermoplastic fibers.

WO-A-9963922 describes an absorbent structure comprising a composite layer for acquisition and distribution of the body fluids and one or more absorbent layers. The various layers are formed using the air-laid technique, i.e. by depositing a web of short fibers on a fabric through which there passes a stream of air sucked in on the side of the fabric opposite to the side on which the fibers are deposited. The absorbent layer consists of cellulose fibers, superabsorbent polymer powders and a binding agent for consolidating the web. The superabsorbent polymer powders are mixed with the fibers before deposition on the fabric being formed. The binding agent is sprayed onto the web of fibers formed on the fabric being formed and is then polymerized by passing the semifinished product into one or more ovens.

The webs forming the acquisition and distribution layer are also formed using the air-laid technique and are consolidated by means of the addition of a binder which is thermally polymerized. Alternatively the webs are consolidated by means of melting of thermoplastic fibers present in the mixtures used for the formation of the said webs. Joining together of the various layers is performed similarly using hot cross-linked binding agents or by means of partial melting of the thermoplastic fibers.

U.S. Pat. No. 5,695,486 describes a composite absorbent structure formed using air-laid techniques in which the webs are consolidated with the addition of binders.

The formation of layers of nonwoven fabric using air-laid techniques has the advantage of allowing the introduction of powders of a superabsorbent polymer (SAP), which is mixed thoroughly with the fibers before formation of the web. The superabsorbent polymers allow the capacity for absorption of fluids by the absorbent layer to be increased considerably. However, this technique also has some drawbacks, including the complexity of the plant necessary for this type of production and the need to use binders in order to consolidate and join together the fibers to form a layer of sufficient mechanical strength. Alternatively thermoplastic fibers and hot calendering systems must be used. The fiber consolidating techniques are a critical aspect, in particular in view of the fact that the fibers used for the formation of the webs using the air-laid technique are very short. The article obtained has a relatively low mechanical strength which constitutes a drawback during the subsequent stages of conversion of the semifinished article for the production of the finished article. Moreover, the use of short fibers oriented in a random manner involves a further problem consisting of the non-optimum distribution of the liquids in the absorbent layer. In fact, the body liquids are released in a fairly restricted zone onto the top sheet of the absorbent article. From here they pass into the underlying absorbent layer of an article which has a longitudinal dimension greater than the transverse dimension. When the fibers are distributed in a random manner, the liquid spreads in the absorbent layer, creating a wet zone with an approximately circular-extension and then reaches the lateral edge zones of the article without impregnating the portions of absorbent material closest to the longitudinal ends, i.e. furthest from the point of entry of the liquid. This results, on the one hand, in the absorbent material not being used in large zones of the article and, on the other hand, in the risk of the liquid escaping laterally.

EP-B-0168196 describes a different method for distributing particles of superabsorbent polymer or powders in a layered article for the production of sanitary napkins. According to this method, the particles are deposited on the top surface of a web and a second web is applied above them. The two webs are joined together by means of embossing, such that the particles remain trapped between the two webs. Distribution of the particles inside the fibrous structure of the strip-like material is therefore not obtained.

### OBJECTS AND SUMMARY OF THE INVENTION

The object of the present invention is the manufacture of an article in a continuous sheet, which overcomes at least partly one or more of the drawbacks of traditional articles.

More particularly, the object of the present invention is the manufacture of a composite structure, to be used as a semifinished product in the production of sanitary napkins, diapers or other similar articles, which can be produced using a simple production line, in which powders or in any case particles of one or more superabsorbent polymers are present, which has a limited thickness, which does not require complex techniques for joining together the various components which form it and which has a high absorption capacity at the same time as a high mechanical strength and a high capacity for distributing correctly and in the most uniform manner possible the liquids in the absorbent mass, using in an optimum manner the volume of the absorbent material, avoiding lateral loss of liquids from the article in which the structure is used.

These and further objects and advantages which will become clear to persons skilled in the art from reading of the text which follows are achieved essentially with an absorbent structure comprising: a first acquisition and distribution layer formed by a nonwoven fabric consisting of non-absorbent fibers; a second absorbent layer formed by a nonwoven fabric consisting of absorbent fibers and comprising particles of at least one superabsorbent polymer distributed in the nonwoven fabric, in which at least said second layer is formed by staple fibers, preferably oriented in a preferred direction of orientation.

In practice, it is advantageous to envisage that at least the second layer is formed by a nonwoven fabric formed by one or more consolidated card webs, in which the particles of superabsorbent polymer are distributed. The consolidating or bonding of the web or webs is obtained preferably by means of mechanical or hydraulic entanglement.

The use of an absorption layer comprising in combination a structure formed by staple fibers, thus fibers of very great length, and a distribution of particles of at least one superabsorbent polymer allows an absorption capacity to be obtained, equal to or greater than those which can be obtained with webs of cellulose fibers formed by means of the air-laid technique with a lower gram weight and a strength much greater than the latter, owing to the greater length of the fiber. If the second layer also has an orientation of the fibers in a preferred direction, which is typically the machine direction, i.e. the direction in which the semifinished product advances during production, the advantage of an improved distribution of the liquids in the absorbent layer is also obtained.

According to the techniques known prior to the present invention it was not possible to introduce particles of superabsorbent polymers into a structure formed by staple fibers, i.e. fibers of considerable length, typically obtained by means of carding. The particles of superabsorbent polymers, in fact, are typically mixed with the cellulose fibers of limited length used for the production of webs by means of the air-laid technique. According to a particular development of the present invention, the first and second layer are joined together preferably by means of entanglement. Moreover, the second layer is advantageously formed by a layer of nonwoven fabric formed by one or more card webs joined and bonded together preferably by means of the same entanglement process with which joining together of the two layers is obtained.

In the context of the present description and the accompanying claims, the term “entanglement” is understood as meaning a process for consolidating a web of fibers and/or joining together two or more webs of fibers and/or one or more webs of fibers and one or more layers of nonwoven fabric and for consolidating or bonding the fibers by means of a mechanical or hydraulic treatment, with which the fibers lying in the individual layers or webs are partly deviated from their original lie so as to form a bundle of consolidated fibers and/or join together their layers or webs of superimposed fibers, by means of penetration, into the bottom web, of some at least of the fibers of the upper web.

The entanglement may be of the mechanical type (mechanical needling), i.e. a needle punching process. In this case, suitably shaped needles are made to penetrate through the superimposed layers of fibers, conveying with them the fibers during penetration into the textile material. This technology is known per se and applied to the production of nonwoven fabrics for various applications, as described for example in the U.S. Pat. Nos. 5,454,145; 5,511,294; 5,548,881 and 4,935,295.

Preferably, however, a hydraulic entanglement or hydroentangling process, also called “spunlacing”, is used. According to this known technique, the material to be consolidated and/or the layers to be joined together are acted on by thin jets of high-pressure water generated by a suitable arrangement of nozzles. The jets of water exert on the fibers of the treated layers an effect similar to that of the mechanical needles. This entanglement technique is described for example in the U.S. Pat. Nos. 3,485,706; 3,493,462; 3,620,903; 4,665,597; 4,623,575; 4,805,275; 5,353,485; 5,475,903; 5,009,747; 6,063,717; 6,163,943 and 6,200,669.

A composite structure according to the invention, which has a second layer formed by staple fibers together with particles of superabsorbent polymer has considerable advantages. The particles of superabsorbent polymer impart a high absorption capacity to the second layer which constitutes, in the end product which is formed with the structure according to the present invention, the internal absorbent layer (or one of the internal absorbent layers). Moreover, the use of staple fibers provides the structure with a high mechanical strength, with the consequent advantages during the subsequent conversion process for obtaining the finished articles.

A composite structure according to the invention eliminates the need to use, during the stage of production of the finished article, i.e. the diaper or sanitary napkin or the like, two separate strip-like materials, one for forming the absorbent core and the other for forming the acquisition and distribution layer. When the layers are combined and consolidated using the mechanical entanglement or hydraulic entanglement techniques, the further advantage is achieved of avoiding the use of binders or of melting the thermoplastic fibers of the layers forming the composite structure. The presence of powders or particles of superabsorbent polymer allows the absorption capacity of the material to be increased and makes it particularly suitable for the production of sanitary napkins and diapers, although using layers of textile fibers of limited thickness. The use of webs of fibers consolidated and joined together by means of entanglement results in a particularly thin finished product.

The use (preferably for both the layers of the structure) of fibers with a length of between about 15 and 60 mm, classified as “staple fibers”, allows high mechanical strengths to be obtained, useful for preventing breakage of the material especially during the subsequent conversion stages, along the converting lines, for the production of the finished products. Preferably, fibers with a length of between about 30 and 60 mm will be used.

A further improvement of the present invention is based on the surprising realization that it is possible to cause penetration and retention, in the layer of absorbent fibers, of the particles of superabsorbent polymer without the latter being retained also in the layer of non-absorbent fibers, i.e. in the acquisition and distribution layer. This makes it possible to produce the structure according to the invention performing first combination, by means of entanglement, of the two webs and then proceeding with application of the superabsorbent polymer.

The penetration of the superabsorbent polymer may be obtained for example by passing the composite material formed by the two previously combined layers—and on which the superabsorbent polymer powder has been distributed—through an alternating electric field, the field lines of which pass through the composite material. Alternatively, the particles of superabsorbent polymer are made to penetrate into the second layer, i.e. into the layer of absorbent fibers, before combining the latter with the first layer.

Advantageously, in this case also, an alternating electric field may be used to obtain the penetration and uniform distribution of the particles in the layer of fibers. In practice, in this case a web of staple fibers is produced, for example by means of a carding process. Then the web is consolidated, for example by means of hydraulic entanglement and dried, forming a layer of nonwoven fabric. A suitable quantity of powder of a polymer or mixtures of superabsorbent polymers is then deposited on said web and is subsequently passed into an alternating electric field in order to cause penetration of the particles or powders into the layer. By using an alternating electric field it is possible to obtain a very uniform and complete distribution of the particles in the entire volume of the nonwoven layer, avoiding consequently the formation of concentrations of particles which, during use of the article obtained with the structure according to the invention, may produce large agglomerations of gel formed by the polymer which has absorbed the liquids. The consolidating advantageously takes place prior to application of the particles of superabsorbent polymer to prevent the latter being lost during the entanglement stage or even before entanglement, owing to the loss of retention inside the web, as a result of the limited consistency of the latter. Moreover, the application of the particles of superabsorbent polymer must necessarily take place after bonding when the latter is obtained by means of hydraulic entanglement.

It is already known to use alternating electric fields to obtain the penetration of powders through fiber structures. For example, WO-A-9922290 describes a method for uniformly filling, throughout its thickness, a fiber structure with powders of polymerizable resins for the purpose of producing a composite material formed by a matrix of polymerized resin reinforced with fibers, for example carbon or glass fibers. This known method is therefore used conventionally to obtain a uniform distribution of powders throughout the thickness of the fiber structure, so as to then perform polymerization in order to obtain the polymerized resin matrix.

It has now been established that this method may also be used for the manufacture of a structure according to the present invention, where it was also found in particular that, under the effect of the alternating electric field, the powders of superabsorbent polymer penetrate and remain incorporated in the absorbent layer, while they do not penetrate into the acquisition and distribution layer. It was even found to be possible to distribute the superabsorbent polymer powders on the external surface of the acquisition and distribution layer and then convey the powders through said layer until they penetrate into the absorbent layer. The superabsorbent polymer powders are not retained by the acquisition and distribution layer.

In practice, according to an aspect of the invention, in order to obtain the abovementioned composite structure, a method comprising the following stages is therefore envisaged:
preparing a first layer formed by non-absorbent fibers;
producing a web of absorbent fibers so as to form a second layer;
arranging alongside each other the first layer and the web intended to form the second layer;
joining together the first layer and the web forming the second layer by means of entanglement, so as to form the composite strip-like material;
causing penetration and retention, in the second layer, of particles of superabsorbent polymer.

The first layer of non-absorbent fibers may be formed by a simple or multiple web of non-bonded fibers, for example supplied from a carding machine or other textile machine which produces a web of fibers, preferably oriented in a preferred direction. Alternatively, the fibers of the first layer may be already consolidated or bonded. Namely, the first layer used in the first stage of the process may be formed by a bonded nonwoven fabric, for example and preferably bonded by means of entanglement. In this case also it will preferably have a predominant orientation of the fibers in a preferred direction. This orientation, which can be obtained for example with a carding machine, improves the function of distribution of the liquids from the outside of the absorbent article towards the core of the article itself. The orientation is preferably in the sense of the machine direction, i.e. in the direction of feeding of the web being formed. In the finished product the acquisition and distribution layer is arranged so that the direction of preferred orientation of the fibers is parallel to the longitudinal extension of the finished product. Thus, the body liquid which accumulates in an approximately central zone of the absorbent material is distributed over the longitudinal extension thereof with a consequent improved use of the absorbent core.

According to this first mode of implementation of the method according to the invention, the second layer of fibers is formed by a (single or multiple) web of non-bonded fibers produced directly upstream of the stage for combination with the first layer. Said web is formed by a random or preferably oriented distribution of fibers, i.e. with a preferred orientation of the fibers, also in this case in the machine direction. This favors a homogeneous absorption of the liquids throughout the volume of the textile layer.

As mentioned, the method for production of the composite layer according to the present invention may envisage:
distributing on a first side of the composite strip-like material the particles of superabsorbent polymer;
applying, through the composite strip-like material, an alternating electric field so as to convey the particles of superabsorbent polymer into the second layer.

If consolidating or bonding of the fibers and joining of the layers forming the composite structure takes place by means of hydraulic entanglement, a drying stage prior to application of the particles of superabsorbent polymer after hydraulic entanglement is envisaged.

According to a different mode of implementation of the method according to the invention, the first and the second layer are consolidated before being combined and the powders or particles of superabsorbent polymer are introduced into the second layer, before combining together. The first and/or the second layer may be obtained by means of consolidating of a single or multiple card web. Consolidating or bonding may takes place by means of a hydraulic entanglement process in which case, prior to insertion of the particles of superabsorbent polymer, drying is suitably performed.

The fibers of the first and second layer have, for example, a count of between about 1 and 15 dtex. Preferably the fibers of the second layer have a count of between about 1 and 2 dtex, while for the first layer fibers with a count preferably of between 8 and 12 dtex are used. The fibers of the second absorption layer are therefore substantially thinner than the fibers of the acquisition and distribution layer.

The first layer may have a weight per unit of surface area preferably less than that of the second layer. For example, the base weight, i.e. the weight per unit of surface area of the first layer may be between about 20 and 120 g/m², while for the second layer it may be between about 40 and 200 g/m², excluding the proportion of the superabsorbent polymer. The latter may constitute from 5 to 50% of the weight of the second layer, i.e. may range between 2-10 and 20-100 g/m². The composite structure therefore has a weight per unit of surface area of between 62 and 320 g/m² before introduction of the superabsorbent polymer.

The first layer, constituting the acquisition and distribution layer, may be formed, for example, by polyester fibers, polypropylene fibers or other hydrophilic or hydrophobic non-absorbent fibers. When fibers of hydrophobic material are used and joining together of the two layers occurs by means of hydraulic entanglement the fibers of the first layer are conveniently treated with agents which make them hydrophilic so as to allow easy penetration of the water jet from the nozzles which perform hydraulic entanglement.

The fibers of the second web may be viscose fibers, cellulose fibers or in any case absorbent fibers.

It is also possible for part of the first and/or second web, or an intermediate layer between them to be formed by a web of bonded or non-bonded cellulose (sheet of porous or non-porous paper). Moreover, environmentally friendly bonded or non-bonded fibers may be inserted in the first and in the second web.

The entanglement is suitably performed so as to provide the absorbent layer with a high degree of compactness, while the first layer is preferably less compact. In order to obtain a different compactness between the first and the second layer, while subjecting both the layers to the same (hydraulic or mechanical) entanglement process, it is advantageous to choose the fibers forming the two layers so that they tend to compact in the second layer and not compact in the first layer.

From this point of view, the choice of viscose for formation of the second layer and polyester for the first layer is optimal since the viscose fibers tend to compact as a result of the effect of entanglement, while the polyester fibers do not tend to compact, but reassume their original bulkiness after the entanglement treatment. This allows a rapid outflow of the body liquids through the first acquisition and distribution layer, on account of the high empty volume which remains between the fibers which form this layer. The low density of the acquisition and distribution layer is advantageous also because it avoids retention of the particles of superabsorbent polymer. Vice versa, the absorbent layer has a greater compactness so as to retain suitably the particles of superabsorbent polymer and reduce the overall volume of the finished product. When body liquids accumulate in this layer, the superabsorbent polymer swells and in any case creates the volume sufficient for storing the said liquids.

The polyester fibers, which are hydrophobic in nature, are treated beforehand so as to acquire hydrophilic properties and allow, on the one hand, as mentioned, easy flow of the water jets in the case of hydraulic entanglement and, on the other hand, a rapid outflow.

### BRIEF DESCRIPTION OF THE DRAWINGS

The invention will be better understood with reference to the description and attached drawing which shows possible non-limiting examples of embodiment of the invention. More particularly, in the drawing:

FIG. 1 shows a very schematic side view of a production line for the manufacture of a composite material according to the invention;

FIG. 2 shows a schematic and simplified cross-section of the composite material;

FIG. 3 shows a schematic side view of a slightly modified embodiment of a production line, similar to FIG. 1;

FIG. 4 shows a schematic side view of a production line in which introduction of the particles of superabsorbent polymer takes place before joining of the two layers.

### DETAILED DESCRIPTION OF THE PREFERRED EMBODIMENTS OF THE INVENTION

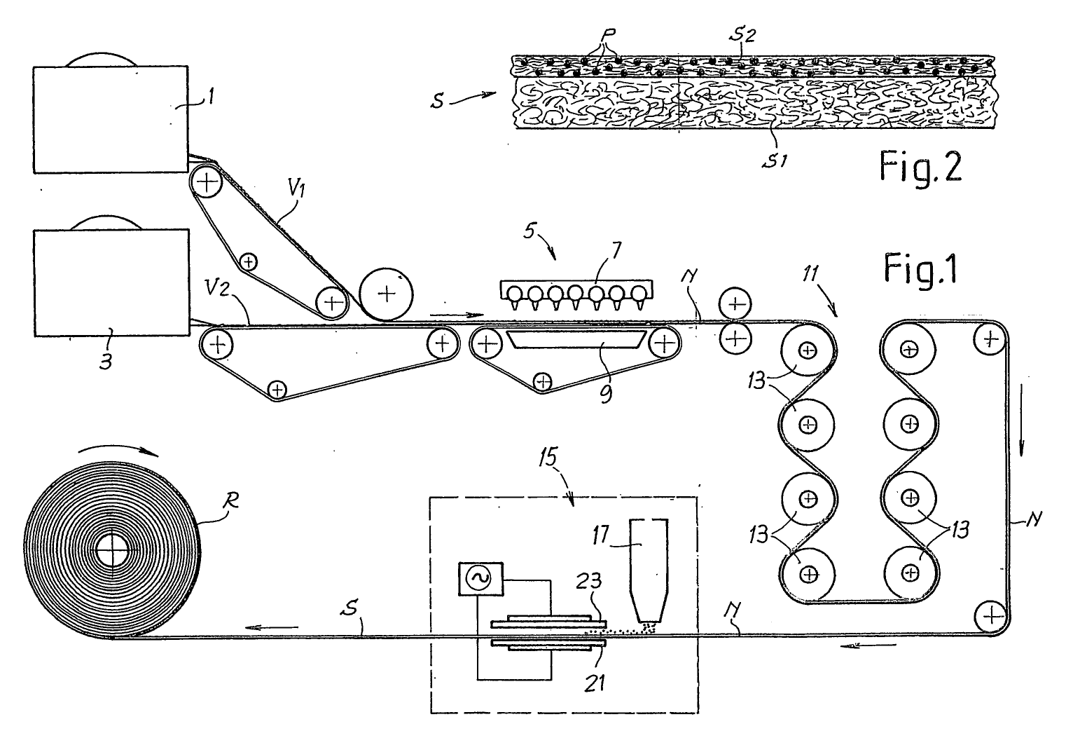

FIG. 1 shows schematically a first embodiment of a production line for producing a structure according to the invention. 1 and 3 denote generally two carding machines or other textile machines able to produce a web of unconsolidated, i.e. non-bonded fibers. Instead of a carding machine, a forming system of the aerodynamic type, a random carding machine, or so-called rando webber, a so-called cross-lapper or other suitable machine may be used.

The expression “web of unconsolidated or non-bonded fibers” is understood as meaning a (single or multiple) web of fibers formed by fibers, bonding of which is generally due exclusively to the intrinsic adhesive power of each individual fiber, without the addition of binders and/or the provision of a bonding treatment. A web of fibers differs, in this sense, from a nonwoven fabric. “Nonwoven fabric” is understood as meaning (according to DIN 61210) a flexible textile product obtained by means of bonding of webs of fibers. The bonding may be achieved in the case of the present invention, mechanically for example by means of needle punching or hydroentanglement.

More particularly, the textile machine denoted by 1 is preferably a machine which forms a web of oriented fibers, i.e. a web where the fibers are arranged principally in a main direction which coincides with the machine direction, i.e. with the direction of feeding of the web being formed. It will therefore consist preferably of a carding machine. The textile machine denoted by 3 forms, on the other hand, preferably a non-oriented web, i.e. a web in which the fibers are arranged in an entirely random manner or in more than one direction and therefore not in a single preferred direction. V1 and V2 denote the two webs produced by the two machines 1 and 3.

The two webs V1 and V2 are superimposed on each other along the feeding path and are then conveyed to a station performing consolidation, by means of a mechanical or hydraulic treatment for consolidation, i.e. bonding the fibers of the two webs. The consolidation station is denoted generally by 5. In this example of embodiment, it comprises a hydroentangling unit, with a series of upper nozzles 7 and a water collection unit 9 arranged underneath the web feeding path. At the exit from the consolidation station the two webs V1 and V2 form a single bonded structure forming a strip-like material N composed of two layers: the first layer formed by the web V1 and the second layer formed by the web V2. The effect of the high-pressure water jets which strike the webs V1, V2 passing through the station 5 is dual in nature: on the one hand, the two webs are joined together and on the other hand the fibers of the individual webs are bonded.

Downstream of the station 5, the strip-like material N is made to pass into a drying station, generally denoted by 11 and comprising (in the example illustrated) a series of heated rollers 13. In this station the residual water retained by the strip-like material N is eliminated and at the exit from the drying station the strip-like material N is substantially dried.

Downstream of the drying station there is a station 15 where the superabsorbent polymer is introduced into the layer formed by the web V2. In the example shown, in this station the strip-like material N enters with the layer formed by the web V2 directed upwards. A hopper 17 deposits onto the free surface of this layer a superabsorbent polymer powder which is then made to penetrate into the layer itself in the manner described below. The quantity of polymer applied per unit of surface area of the structure S may vary, for example, from 5 to 50% by weight of the weight per unit of surface area of the web V2 forming the second layer of the strip-like material N. The quantity may be chosen for example depending on the finished product which is intended to be made with the composite structure.

Superabsorbent polymers for these uses are known to persons skilled in the art and are commercially available. These include, for example, the polyacrylate marketed under the name IM1000 by Hoechst-Celanese, USA, Sanwet® produced by Sanyo Kasei Kogyo Kabushiki, Japan, Favor® produced by Stockhausen, USA, the polymers marketed by Chemdal, USA, under the name ASAP®, the polymer Sumika Gel®, produced and marketed by Sumitomo Kagaku Kabushiki Haishi, Japan. Other suitable products are known to persons skilled in the art and some of them are mentioned in WO-A-9963922 and in EP-B-0168196.

The strip-like material is then passed between two armatures 21, 23 between which an alternating electric field is generated, for example as described in WO-A-9922920. This alternating electric field causes conveying of the superabsorbent polymer particles deposited on the upper surface of the strip-like material N inside the material itself. It has been found that the superabsorbent polymer powders are retained by the second layer formed by the web V2, while they are not retained by the layer formed by the web V1. At the exit from the station 15, therefore, a structure S which in cross-section has the appearance shown schematically in FIG. 2 is obtained. It consists of a first layer S1 formed essentially by the web V1 bonded and joined to the adjacent web V2 which forms a layer S2. The layer S2 has a content of superabsorbent polymer powder retained by the meshwork of fibers forming the layer itself. The polymer is schematically denoted by P.

The structure S produced continuously with the plat described is wound so as to form rolls or reels R intended to be used as a semifinished starting material in lines for the production of sanitary napkins, diapers and other similar articles.

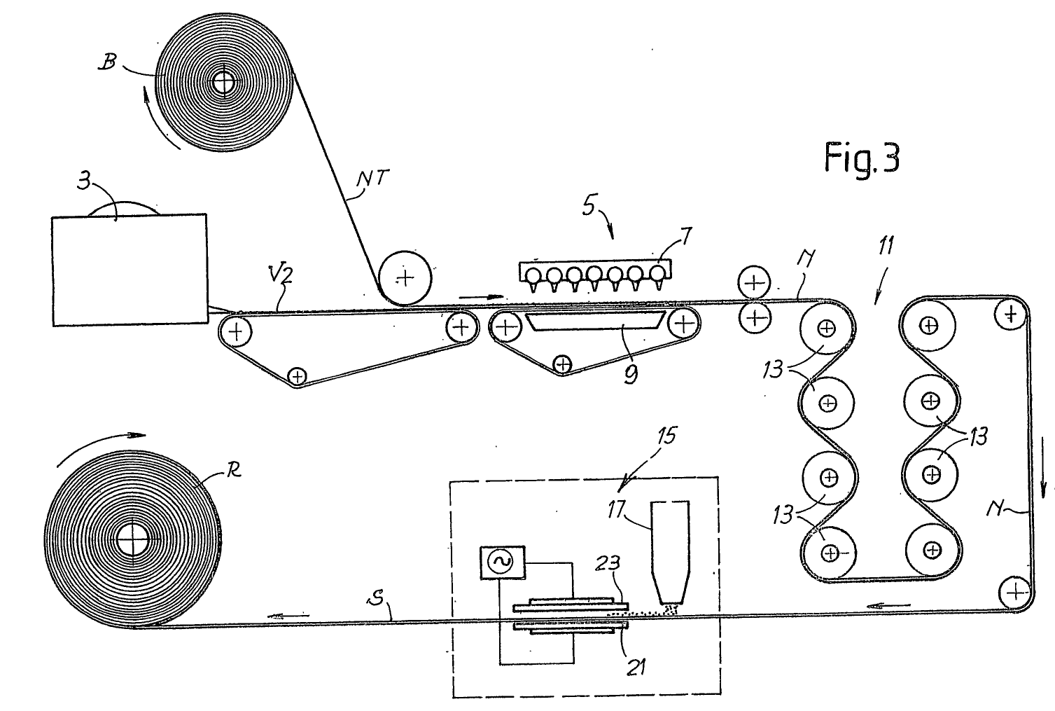

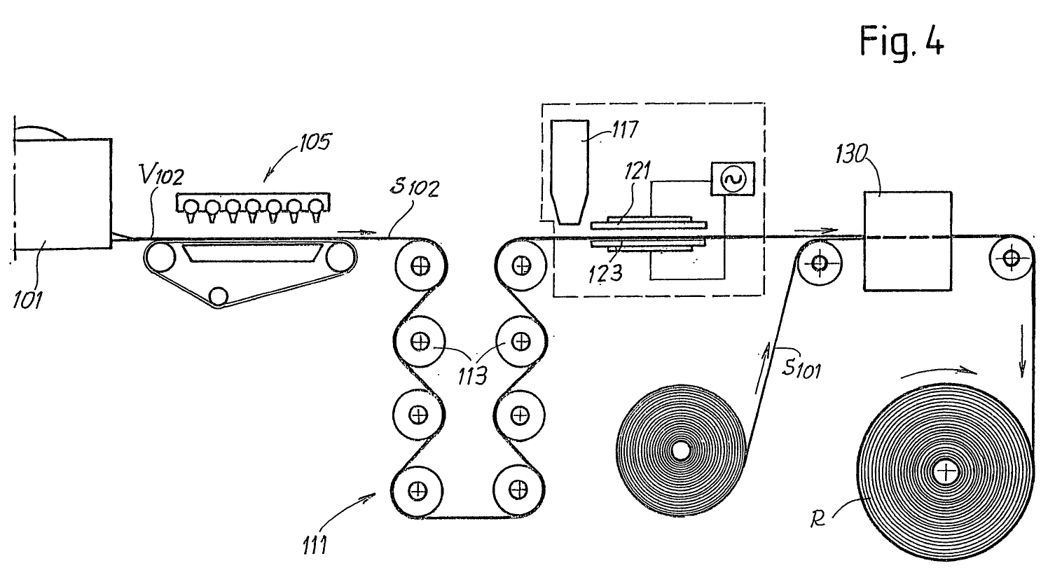

FIG. 3 shows a modified embodiment of the plant shown in FIG. 1. The same numbers denote parts which are the same or correspond to the parts of the plant in FIG. 1. The difference between the two solutions consists in the fact that the layer S1 in this case is not produced from a web of non-bonded fibers supplied by a textile machine, especially a carding machine. Instead, along the feeding path the web V2 intended to form the second layer of the composite structure is combined with a nonwoven fabric NT supplied by a reel B formed previously in a different plant. The nonwoven fabric intended to form the first layer S1 of the structure S may be a materials which has been entangled or also consolidated in another way. The advantage of this solution consists in the fact that, in the consolidation station 7 for hydroentangling the two components intended to form the layers S1 and S2, the density of the layer of nonwoven fabric NT remains substantially unvaried. It is therefore possible to control independently the density of the two layers S1, S2 of the final structure S and obtain an acquisition and distribution layer S1 with a substantially smaller density (greater empty spaces between the fibers) compared to the absorbent layer S1.

FIG. 4 shows a modified production line for implementing a method in accordance with a different embodiment of the invention. In this case, 101 denotes a textile machine, for example a carding machirie, which forms a web V102 of staple fibers oriented with a preferred orientation in the machine direction. The web V102 is consolidated in a consolidation station 105 by means of hydroentanglement so as to obtain a layer S102 consisting of a web of consolidated fibers, i.e. a nonwoven fabric. The layer thus consolidated is then passed through a drying station 111 formed by a series of heated rollers 113 in order to eliminate the residual water retained in the material forming the layer S102. Then, downstream of the drying station 111, particles of superabsorbent polymer or a mixture of superabsorbent polymers are distributed uniformly on the upper surface of the layer S102, by means of a hopper 117. Downstream of the latter there is provided a pair of armatures 121, 123, between which an alternating electric field is generated, similar to that described with reference to the armature 21, 23 of the preceding embodiment. The alternating electric field causes the penetration of the particles throughout the thickness of the layer S102.

Downstream of the armatures 121, 123, the layer S102 is combined with a layer S101 which, in this example of embodiment, is a layer of nonwoven fabric which is supplied from a supply roll, but which could be a layer of nonwoven fabric produced along the line, for example by means of a carding machine or other textile machine, in accordance with that described with reference to the preceding embodiments.

A joining station 130, consisting for example of an ultrasound welding unit of a type known per se, is provided downstream of the zone for combining the two layers S102, S101. With these welding units it is possible to perform spot-welding, heating the layers S102 and S101 in defined zones. Either one or both these layers contain at least a fraction of thermoplastic fibers which melt at the temperature to which they are heated in the joining station 130, so as to ensure spot-welding between the two layers.

Alternatively, it is possible to envisage a different system for joining together the layers S102 and S101, for example by means of gluing, distributing onto either one or both the layers an adhesive, for example an adhesive which can be heat-activated. Then the two layers are then passed into a calender consisting of a pair of heated cylinders, in order to activate the adhesive. Alternatively, the heated-cylinder calender may also be used for causing partial melting of the thermoplastic fibers present in the mixture forming either one or both the layers. Welding may also be performed spotwise in this case by means of incision of at least one of the cylinders of the calender, namely by providing it with a series of protuberances or projecting points.

At the exit from the joining station 130, in this case also, a product which has a cross-section similar to that shown in FIG. 2—except for the different type of joint between the two layers—is obtained. In this case, the advantage of the complete elimination of the adhesives and/or of melting—including only partial melting—of the fibers forming the two layers is lost.

1. An absorbent structure comprising: a first acquisition and distribution layer formed by a nonwoven fabric of substantially non-absorbent fibers; a second absorbent layer formed by a nonwoven fabric of absorbent fibers and comprising particles of at least one superabsorbent polymer distributed in the nonwoven fabric, wherein:
said first layer and said second layer are formed by a nonwoven web of carded staple fibers, consolidated by entanglement;
and said first layer and said second layer are joined together by entanglement.

2. Structure as claimed in claim 1, wherein said absorbent fibers have a length of between about 15 and 60 mm.

3. Structure as claimed in claim 1 or 2, wherein said non-absorbent fibers have an overall length of between about 15 and 60 mm.

4. Structure as claimed in claim 1, wherein the absorbent fibers of the second layer have a count of between about 1 and 15 dtex.

5. Structure as claimed in claim 1, wherein the non-absorbent fibers of the first layer have a count of between about 1 and 15 dtex.

6. Structure as claimed in claim 1, wherein the first layer has a weight per unit of surface area of between about 20 and 120 g/m².

7. Structure as claimed in claim 1, wherein the second layer has a weight per unit of surface area, excluding the superabsorbent polymer, of between about 40 and 200 g/m².

8. Structure as claimed in claim 1, wherein the superabsorbent polymer is present in a quantity by weight of between about 5 to 50% of fiber weight forming the second layer.

9. Structure as claimed in claim 1, wherein said first layer and said second layer are joined together by hydraulic entanglement.

10. Structure as claimed in claim 1, wherein said first layer and said second layer are joined together by mechanical entanglement.

11. Structure as claimed in claim 1, wherein said nonwoven fabric of the first layer is consolidated prior to joining with the second layer.

12. Structure as claimed in claim 11, wherein the fibers forming the first layer are bonded together to form a consolidated nonwoven fabric and said consolidated nonwoven fabric is joined by entanglement to the second layer.

13. Structure as claimed in one or more of the preceding claims, claim 1, wherein the fibers of the second layer have a preferred orientation in one direction.

14. Structure as claimed in claim 1, wherein the fibers of the first layer have a preferred orientation in one direction.

15. Structure as claimed in claim 1, wherein said first layer has a thickness of between about 0.15 and 1.5 mm.

16. Structure as claimed in claim 1, wherein said second layer has a thickness of between about 0.5 and 2 mm.

17. Structure as claimed in claim 1, wherein the non-absorbent fibers of the first layer are selected from polyester fibers, polypropylene fibers, cellulose fibers or a mixture thereof.

18. Structure as claimed in claim 1, wherein the absorbent fibers of said second layer are viscose fibers.

19. Structure as claimed in claim 1, wherein the structure is in a form of a strip-like material wound into a roll.

20. Structure as claimed in claim 1, wherein said first layer has a compactness which is less than that of said second layer.

21. A method for producing an absorbent, composite, strip-like material, comprising a first layer for acquisition and distribution of a liquid and a second absorbent layer for absorbing and retaining said liquid, including the steps of:
forming said first layer of non-absorbent fibers;
producing a web of absorbent staple fibers to provide the second layer;
arranging alongside each other the first layer and the second layer;
joining together the first layer and second layer by entanglement so as to form the composite strip-like material;
distributing particles of at least one superabsorbent polymer on one side of the composite strip-like material;
applying an alternating electric field through the composite strip-like material so as to convey and introduce said particles of superabsorbent polymer into said second layer.

22. Method as claimed in claim 21, further comprising forming at least said second layer with a preferred orientation of the fibers.

23. Method as claimed in claim 21 or 22, wherein the particles of superabsorbent polymer are distributed over a free surface of the second layer.

24. Method as claimed in claim 21, wherein said non-absorbent fibers have a length of between about 15 and 60 mm.

25. Method as claimed in claim 21, wherein said absorbent fibers have a length of between about 15 and 60 mm.

26. Method as claimed in claim 21, wherein said web of absorbent fibers is a web of fibers carded and not consolidated before joining with the first layer.

27. Method as claimed in claim 21, wherein said first layer is formed by a nonwoven fabric consolidated before joining with said second layer of absorbent fibers.

28. Method as claimed in claim 21, wherein said first layer is formed by a web of fibers not consolidated before joining with said web of absorbent fibers, the first layer and the second layer being consolidated and joined together in a single entanglement operation.

29. Method as claimed in claim 21, wherein said first layer is formed with a preferred orientation of the fibers.

30. Method as claimed in claim 21, further comprising joining together said first layer and said web of absorbent fibers intended to form the second layer, by hydraulic entanglement, and drying the strip-like material before applying the particles of superabsorbent polymer.

31. Method as claimed in claim 20, further comprising joining together said first layer and said web of absorbent fibers intended to form the second layer, by mechanical entanglement.

32. Method as claimed in claim 20, wherein said second layer is formed with fibers having a count of between about 1 and 15 dtex.

33. Method as claimed in claim 21, wherein said first layer is formed with fibers having a count of between about 1 and 15 dtex.

34. Method as claimed in claim 21, wherein said first layer has a weight per unit of surface area of between about 20 and 120 g/m².

35. Method as claimed in claim 21, wherein the web of absorbent fibers intended to form the second layer has a weight per unit of surface area of between about 40 and 200 g/m².

36. Method as claimed in claim 21, wherein a quantity of the particles of superabsorbent polymer are present in an amount between about 5 to 50% by weight compared to weight of the fibers forming said second layer prior to introduction into said second layer.

37. A method for producing an absorbent, composite, strip-like material, comprising a first layer for acquisition and distribution of a liquid and a second absorbent layer, for absorbing and retaining said liquid, including steps of:
preparing said first layer;
preparing said second layer;
distributing particles of at least one superabsorbent polymer on one side of said second layer;
after distribution of said particles on said second layer, inserting said particles of said at least one superabsorbent polymer in said second layer by applying an alternating electric field through said second layer;
joining together said second layer with said first layer.

38. Method as claimed in claim 37, wherein said joining together of said first layer and said second layer is by spot-welding.

39. Method as claimed in claim 38, wherein said joining together of said first layer and said second layer is by ultrasound welding.

40. Method as claimed in claim 38 or 39, wherein at least one of said first layer and said second layer includes, a percentage of thermoplastic fibers.

41. Method as claimed in claim 37, further comprising applying an adhesive between said first layer and said second layer and joining said first layer and second layer by gluing.

42. Method as claimed in claim 41, wherein said adhesive is a heat-sensitive adhesive.
